(19) United States
(12) Patent Application Publication
(10) Pub. No.: US 2020/0138570 A1
(43) Pub. Date: May 7, 2020

(54) HEART IMPLANT

(71) Applicant: coramaze technologies GmbH, Hilden (DE)

(72) Inventors: Youssef BIADILLAH, Munchen (DE); Laura FIGULLA, Munchen (DE); Stefan Daniel MENZL, Hersbruck (DE); Thomas GERHARDT, Munchen (DE); Niklas Maximilian NATHE, Dusseldorf (DE); Raz BAR-ON, Hadera (IL); Leah KIDNEY, Munchen (DE); Mario HEINTZE, Dresden (DE)

(73) Assignee: coramaze technologies GmbH, Hilden (DE)

(21) Appl. No.: 16/737,968

(22) Filed: Jan. 9, 2020

Related U.S. Application Data
(63) Continuation of application No. PCT/EP2017/000805, filed on Jul. 10, 2017.

(51) Int. Cl. A61F 2/24

(52) U.S. Cl. CPC A61F 2/246; A61F 2220/0008; A61F 2220/0041

(57) ABSTRACT

The invention relates to a heart implant, particularly being configured to reduce or eliminate a heart valve insufficiency after implantation into the heart, comprising a closure element (1) being configured to be positioned within the heart valve annulus, particularly being configured to close or at least to reduce a remaining gap between closing valve leaflets, an anchoring element (2) being attached to the closure element (1) for fixing the implant in the heart, preferably for non-invasive fixing by surface contact between the exterior surface of the anchoring element (2) and an interior surface of a heart lumen, preferably the atrium, wherein the closure element (1) and the anchoring element (2) are connected with a means (1a, 2a) for changing the relative position of the closure element (1) and the anchoring element (2) to each other. The invention furthermore relates to a method of treating a heart valve insufficiency.

### RELATED APPLICATION(S)

This application is a Continuation of PCT Patent Application No. PCT/EP2017/000805 filed on Jul. 10, 2017, the contents of which are incorporated by reference as if fully set forth herein in their entirety.

### FIELD AND BACKGROUND OF THE INVENTION

The invention relates to a heart implant, particularly a heart implant being configured to reduce or eliminate a heart valve insufficiency after implantation into the heart.

Typically, such implants are positioned in such a way that a closure element of the implant is situated in the valve annulus (for example mitral or tricuspid valve) and closes a remaining gap of the closed valve leaflets. For that purpose, the closure element is connected to at least one anchoring element, for example an anchoring cage, being configured to fix the closure element within the heart in the desired position i.e. in the valve annulus preferably to be contacted by the closing valve leaflets.

In a possible embodiment disclosed in the applicants' own prior patent filings the closure element may be formed by an inflatable sheath I membrane that is positioned, preferably coaxially positioned around a central column element, preferably a tubular central column element and fixed to this element at the respective ends of the sheath I membrane to get a fluid tight space around the central column element. Such a fluid tight sheath may be inflated with a fluid. Preferably the areas of attaching the sheath to the central column element also define the upper and lower end of the closure element.

In another embodiment disclosed in the applicants' own prior patent filings the closure element may be also formed of a sheath/membrane being supported by an expanded part of a central column element, preferably tubular central column element. The expanded part may form a scaffold structure, preferably a meshed scaffold structure that supports the sheath from the inside. By expanding the central column element I scaffold structure also the sheath expands in cross section and contacts the underlying scaffold structure formed by the expanded column element. The sheath also here prevents blood from passing through the valve in a closed leaflet state and may also be fluid tight.

The sheath of such a closure element is not necessarily fluid tight from the beginning of implantation. The sheath may have pores for allowing blood to enter the inner space of the closure element but may not allow clotted blood to escape from the inner space of the closure element. The blood may get clotted more and more with time and may close the sheath and thus form the closure element accordingly.

The implant of the invention may comprise any kind of closure element and may preferably be a closure element as described above.

In general and for the purpose of this invention the closure element is a plug that is configured to be positioned within the valve annulus of a valve that is to be treated. This plug will close or at least reduce a remaining gap between the closing leaflets of the valve.

It is known in the state of the art to use an anchoring element secured into the myocardium tissue of the ventricle for fixation of the closure element. Besides this invasive way, other implants provide a less invasive fixation just by contacting the interior wall of the atrium and/or ventricle with the outer surface areas of an anchoring element formed of an expanded cage that is connected to the closure element.

Such cage typically is crimped into a collapsed state for insertion of the entire implant through a catheter into the heart where it is expanded after release from the catheter for fixation purposes. The invention preferably relates to such implants having an expandable, particularly mesh-like anchoring cage formed of preferably interconnected strips for anchoring purposes.

An anchoring cage may also be formed without meshes, particularly just by several side-by-side-lying strips having no interconnection. The invention in general also relates to non-meshed cages and any other suitable anchoring element(s) attached to the closure element for fixation purposes, particularly for non-invasive fixation purposes.

In general and for the purpose of this invention an anchoring element is an anchor configured to fix the implant in the heart, preferably by surface contact only, i.e. without puncturing the myocard.

It is furthermore disclosed in the applicants' own prior patent filings that the central column element, preferably a tubular column element or tube has a lower end and an upper end and is split into several strips at least at the upper end, the strips forming an expandable cage as mentioned, particularly for fixing the heart implant to the atrium of the heart by surface contact between an exterior surface of the expandable cage (the several strips) and an interior atrium surface.

The mentioned positions “lower” and “upper” or directions mentioned in this disclosure are to be understood in the intended position of the implant if it is correctly implanted in the heart. In the heart the atrium is positioned above the ventricle and accordingly the lower end of the closure element faces the ventricle, particularly is positioned in the ventricle and the upper end faces the atrium, particularly is positioned in the atrium if correctly implanted. A middle part of the closure element between the upper and lower end is passing through the valve annulus of the valve that is to be treated, preferably the mitral or tricuspid valve.

The central column element, particularly the tubular central column element preferably the so formed scaffold structure and the strips of an anchoring cage may originate from one single tube by cutting the tubular wall several times, preferably in an axial direction the mentioned strips all start their extension from an annular upper end area of the central column element I scaffold structure and preferably are equally spaced along the circumference of this end. Such a cage may also be formed of strips starting their extension at the lower end of the central column element.

An anchoring element, particularly cage-like element is preferably formed by splitting and merging strips thus forming a half mesh between the points of splitting and merging. This embodiment is also preferred for the invention described in this disclosure.

An anchoring cage having several meshes is formed that way for solely fixing the heart implant to the atrium and/or ventricle of the heart by surface contact between the exterior cage surface and the interior surface of the respective heart lumen (atrium or ventricle). Preferably, the invention relates to an implant having a single anchoring cage only on the atrial side of the closure element.

A cage being formed of several expanded strips originating from a cut tube by radial expansion provides the advantage that the strips may generate a radial force being essentially perpendicular to the axis of extension of the (tubular) attachment element to keep the anchoring cage in place after implantation and expansion. The anchoring cage is sufficiently compliant in radial direction in order to adapt its shape to the atrium.

In general, heart implants for reducing or eliminating a heart valve insufficiency are designed and fabricated in a pre-determined size. Since the heart anatomy varies from patient to patient it is necessary to hold on stock different sizes and to select for surgery the best fitting one.

In addition, it has been found so far that a closure element having a circular cross section is the best compromise to suit different gap sizes and gap shapes between the closed leaflets. Nonetheless, the existing gap is in most cases non-circular and accordingly a non-circular closure element would fit better and would create more uniform forces in the contact area of leaflets and closure element. But using a non-circular closure element is much more demanding in regard to the correct adjustment.

Accordingly it is an object of the invention to improve existing heart implants and to provide an inventive implant that better fits to different heart sizes and particularly may be adjusted for best fit the gap between the closed leaflets.

Even though the application of the implant and method is preferred in regard to humans the implant and method of treatment may be also applied to animals, particularly mammalian animals.

### SUMMARY OF THE INVENTION

The object is solved by a heart implant comprising a closure element being configured to be positioned within the heart valve annulus, particularly being configured to close or at least to reduce a remaining gap between closing valve leaflets, and an anchoring element being attached to the closure element for fixing the implant in the heart, preferably for atraumatic fixing by surface contact between the exterior surface of the anchoring element and an interior surface of a heart lumen, preferably the atrium, most preferred the left atrium, wherein the closure element and the anchoring element are connected with a means for changing the relative position of the closure element and the anchoring element to each other.

Using such a means for changing the relative position of the closure element and the anchoring element to each other provides the ability to perform adjustments between the anchoring element and the closure element, particularly adjustments that change the overall length of the entire implant. In an application for the mitral valve the length is regarded in an axial direction from ventricle through the mitral valve to the atrium. Accordingly it is possible to adjust a given fabricated implant to different heart anatomies, particularly different heart sizes and preferably to different positions of the valve annulus in relation to the top of the atrium.

Particularly for implants that are atraumatically fixed by means of an anchoring cage in the atrium only the position of the anchoring cage is determined by the lumen of the atrium, since such a compliant cage is preferably self-centering in this lumen by the forces exerted from the cage strips to the atrial wall. By using the inventive implant it is now possible to adjust the position of the closure element relative to the anchoring element after the anchoring element is positioned in the lumen of the atrium.

According to a preferred embodiment the means for changing the relative position is a means for changing the axial position of the entire closure element and the entire anchoring element with respect to each other. In this embodiment the closure element may be moved along the afore-mentioned axis of the implant by operating the mentioned means. The position of the closure element may be improved after implantation in order to optimize the position of the closure element in the valve annulus in regards to the axial position. For example, the position of the closure may be amended to have the middle of the closure element surrounded by the valve annulus. In embodiments of the closure element having a circular cross section, particularly being rotationally symmetric, the means for changing the relative position of the closure element and the anchoring element to each other may be configured only to adjust the axial distance between the closure element and the anchoring element.

According to the invention it is also possible that the means for changing the relative position is a means for changing the rotational (or angular) position of the entire closure element and the entire anchoring element with respect to each other. This embodiment is particularly useful for implants having a closure element with a non-circular cross section but not limited to these.

This embodiment provides the possibility to rotate the closure element relative to the anchoring element around the mentioned axis after the anchoring element is fixed in the atrium and may not be moved any more. The closure element may now be rotated for best fit of its shape to the shape of the remaining gap between the closed leaflets.

The invention may also provide a combination of both possibilities to change the relative position of the anchoring element and the closure element. Accordingly, in this combination a single means for changing the relative position may be configured to perform changes in axial and rotational position, particularly simultaneously. It is also possible to provide different means for changing the relative position, i.e. a means for changing the axial position and another means for changing the rotational/angular position. In view of the fact that these means are distinct the two possible adjustments may be performed independently.

A structural embodiment that may provide the means for changing the relative position of the closure element and the anchoring element with respect to each other is formed of an adjustable pair of co-working connectors configured to connect the closure element and the anchoring element. Preferably, the mentioned pair of connectors comprises two connectors that are engageable and disengageable.

For example, the closure element comprises a first connector facing towards the anchoring element, particularly the first connector being configured to be positioned in the atrium of the heart. The anchoring element comprises a second connector facing towards the closure element, particularly the second connector being also configured to be positioned in the atrium of the heart. The means for changing the relative position of the closure element and the anchoring element with respect to each other is formed of these two connectors by one of the two connectors being configured to be axially movable and/or rotatable along and/or around the other connector. Preferably one of the connectors is at least partially put into the other for connection purposes and movable longitudinally and/or around its axis in this configuration.

In order to provide the possibility to disengage the two connectors a trigger handle may be provided to at least one of the two connectors, for example to allow pulling one of the connectors out of the other. Using the trigger a catch or locking mechanism in the pair of connectors may be actuated. The trigger handle may form simultaneously a locking element of the locking mechanism. Such a locking element may have a locking position in which the two connectors are engaged and secured in this situation and may have another un-locking position in which the two connectors are detachable, particularly one of the connectors may be pulled out of the other connector in this position.

In a preferred embodiment the closure element comprises a first central column element, particularly an expandable and/or compressible part of the closure element being formed of the first column element or attached to the first column element, the first connector being positioned at the end of the first column element facing towards the anchoring element. In this embodiment the anchoring element comprises a second central column element, particularly the second central column element at its end facing away from the closure element being split into several strips for forming an expandable anchoring cage, the second connector being positioned at the end of the second column element facing towards the closure element. The cage may be formed as disclosed in the introductory section.

Furthermore in this preferred embodiment the two central column elements may originate from an original single central column element that is divided into two parts. Nonetheless it is also possible to fabricate the two central column elements from two distinct parts. The respective first and second connector may be formed from the same single or the respective distinct element or may be attached to them, for example by any kind a fusion, i.e. welding, soldering or gluing. The respective central column elements may be a tube, slotted tube, particularly expanded slotted tube.

Preferably, and particularly in combination with the mentioned embodiment having column elements, the first and the second connectors may form a nut and screw arrangement, allowing simultaneously an axial and rotational adjustment of the closure and anchoring element relative to each other. In that case one of the connectors forms a screw and the other forms a nut. In this embodiment the two connectors may be brought into a detaching position in which the two connectors are automatically detached. This position is achieved if the two threads of nut and screw disengage.

The connectors may also form a tab I ratchet arrangement allowing axial adjustment independent of rotational adjustment of the closure and anchoring element relative to each other and I or allowing rotational adjustment independent of axial adjustment of the closure and anchoring element relative to each other.

Each pair of connectors may form a ratchet mechanism, preferably a one-way ratchet mechanism, and most preferred a releasable ratchet mechanism having several, preferably equally spaced stop positions. By changing the respective stop position used in the two connectors their axial position may be changed. The invention may allow, in each possible stop position, the ability for the two connectors to rotate against each other.

The ratchet mechanism may be releasable by using a releasing handle at one of the two connectors particularly the one that is inserted into the other. For example the entrance area may be enlarged or a stop member may be retracted using the releasing handle in order to allow the movement of one connector particularly opposite to the one-way direction.

The two connectors may also form a telescopic construction in which one of the connectors is guided in the other. The two connectors may be formed of tubes that fit together. Also here a ratchet mechanism or any other locking mechanism may be realized between the two connectors for example being effective between the two opposite lateral areas of the tubes. One of the two tubes may have a releasing handle for releasing the ratchet. The releasing handle may be simultaneously the locking element of a locking mechanism.

In general and particularly in embodiments allowing only rotational position changes but no change in axial position the two connectors may be connected by a form-closed link, preferably having a rotational symmetry in the two connectors with the rotational symmetry allowing to rotate the two connected connectors against each other. The invention may provide that a rotation is only possibly if a threshold force I torque is overcome, for example to overcome a given friction between the two connectors. Accordingly, unintentional rotation is prevented by friction or any other kind of force link between the two connectors. In a possible embodiment the two connectors may comprise permanent magnets having opposite polarity in the mating surfaces. Such connectors are detachable and if they are attached to each other allow rotation if the friction of the mating surfaces is overcome.

Particularly in all possible embodiments the two connectors and accordingly the closure element and anchoring element may be detachable.

Particularly when allowing the change of rotational position, and maybe also in embodiments of axial position changes that are not self-inhibiting the pair of two co-working connectors may have a stop mechanism, preventing the unintentional movement of the two connectors. Also such a stop mechanism may simultaneously form the locking mechanism that prevents unintentional detachment of the two connectors.

In general, the two connectors may be detached by moving one of the connectors relative to the other connector into a detaching position in which both connectors are automatically detached. Moving the connectors may be possible only after overcoming a threshold force. Detaching may be also done by operating a locking element of a locking mechanism I stop mechanism.

The invention allows to perform a method of treating a heart valve insufficiency by implanting an implant into the heart, the implant comprising a closure element that is positioned within the heart valve annulus and being configured to close or at least to reduce a remaining gap between closing valve leaflets and comprising an anchoring element being attached to the closure element, the implant being fixed in the heart with the anchoring element, preferably by surface contact between the exterior surface of the anchoring element and an interior surface of a heart lumen, preferably the atrium, wherein after setting the implant into the desired place the closure element is adjusted relative to the anchoring element in its axial and I or rotational position for improving the coaptation between the leaflets of the heart and the closure element.

The closure element and the anchoring element may be realized in general as mentioned before in the introductory section.

Preferably, the closure element is connectable or releasably connected to an operating means for operating the means for changing the relative position. For example by rotating or axially moving the closure element the mentioned means for changing the position is indirectly operated by transferring the necessary force or torque via the closure element.

An operating means may be an automatically or manually operated controller, particularly a handle, having force or torque transmitters, preferably wires for moving the mentioned connectors relative to each other.

The operating means may have a length configured to be fed from the proximal end of a catheter through the entire length of the catheter and to the distal end of the catheter. The proximal end of the catheter is understood to be the end lying outside of a treated patient and the distal one is the other end located in the heart. This embodiment ensures that a surgeon can cause the desired change in position of the closure element by operating the operating means from outside the patient. Any change in position may be observed in live X-ray images. For that purpose the closure element may comprise radiopaque materials, radiopaque markers, or a combination of the two.

Preferably the means for changing the position is operable by pulling and/or turning the operating means. The operating means may be formed of a pull or push wire, for example made of metal or of a torque transmitting bendable shaft in order to achieve this.

A surgeon may manually operate the operating means but it is also possible that at the proximal end of the catheter the operating means is connectable I connected to a controller that actively controls the amount of change in position of the closure element, particularly by measuring the number of revolutions performed or measuring a moved distance with the operating means. Such controller may comprise at least one actor to pull/push or turn the operating means.

In a preferred embodiment the operating means (one or more operating elements of it) is disconnectable from the closure element and retractable out of the catheter after the desired position is met. The operating means may also serve to perform or to initiate the expansion of the closure element, particularly if it is not self-expanding after release out of a catheter.

### BRIEF DESCRIPTION OF THE SEVERAL VIEWS OF THE DRAWING(S)

FIG. 1: shows an implant of the invention having a closure element and an anchoring element being detachable connected by a tab link allowing rotation

FIG. 2: shows a bayonet link between the connectors, allowing rotation and detachment of the elements

FIG. 3: shows a magnetic link between the elements, allowing rotation and detachment of the elements

FIGS. 4A-B: show a screw nut arrangement allowing simultaneous rotational and axial position changes and detachment of the elements

FIG. 5: shows a ratchet link allowing independent axial and rotational position changes and detachment of the elements

FIGS. 6A-E: show schematically different types of connections between two connectors

### DESCRIPTION OF SPECIFIC EMBODIMENTS OF THE INVENTION

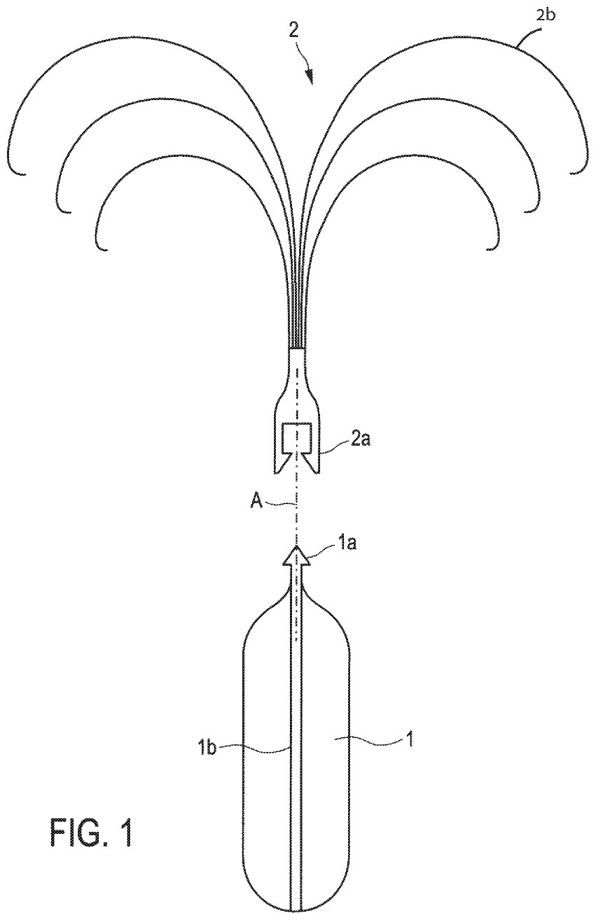

The FIGS. 1 to 5 show an implant having a closure element 1 and an anchoring element 2. The anchoring element 2 is formed of an expandable cage 2 that is shown in the expanded configuration. The cage 2 is positioned above the closure element 1 and intended to fix the entire implant in the atrium of the heart, preferably the left atrium. The closure element 1 is intended to be positioned in the valve annulus of the mitral valve thus forming a plug that serves to close or at least to reduce a remaining gap between the closing leaflets.

The cage 2 comprises several strips 2b that emerge from the second connector 2a upwards, are bent preferably by at least 180 degree, and extend back towards the connector 2a and the closure element 1.

The FIGS. 1 to 5 just show a cross-sectional view. The strips 2a of the anchoring cage 2 will also extend in other planes and not only in the one shown. The strips 2a may comprise not shown split strip regions and merged strip regions forming a mesh construction. The cage 2 is resilient in radial direction, i.e. perpendicular to the central axis A lying in the paper plane and may adapt to the shape of the atrium. Fixation is just done by a form fit or force fit between the atrial wall and the cage 2. In general, the cage 2 is also resilient in the axial direction of axis A due to the fact that the lower strip ends of the cage 2 are free and the strips 2b between the lower end and upper end of the cage 2 are curved. So the lower free strip ends may be moved upwards and the entire cage 2 may be compressed in the axial direction.

The closure element 1 comprises a central column element 1b to which an expandable sheath is attached. The column element 1b is shown in FIG. 1 only but may also apply to all the other figures. The column element 1b is a tube passing through the closure element that forms the plug for preventing regurgitation of blood.

In all embodiments, the closure element 1 comprises a first connector 1a and the anchoring element comprises a second connector 2a. The connectors 1a I 2a may be understood as the axial end part of a column element and may be connected or detached.

FIG. 1 shows a tab link between the two connectors 1a I 2a. If the two connectors are connected they can just be rotated to adjust different rotational I angular positions between closure element 1 and anchoring element 2. A frictional force between the two connectors 1a I 2a may prevent unintentional movement.

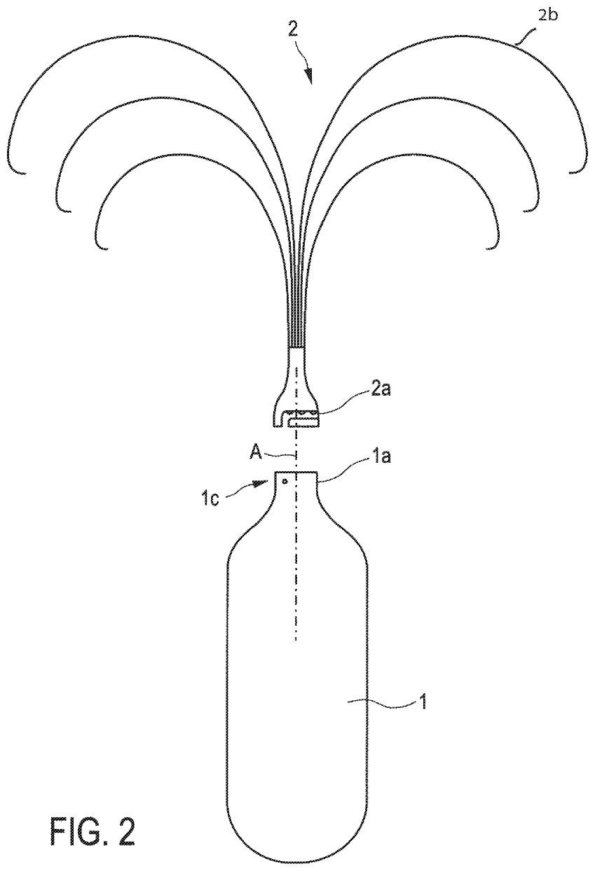

FIG. 2 shows a bayonet link between the connectors 1a and 2a. The two connectors are detachable and if connected may only be rotated. The pin 1c of connector 1a may rest in different distinct positions defined along the circumferentially extending slot of connector 2a.

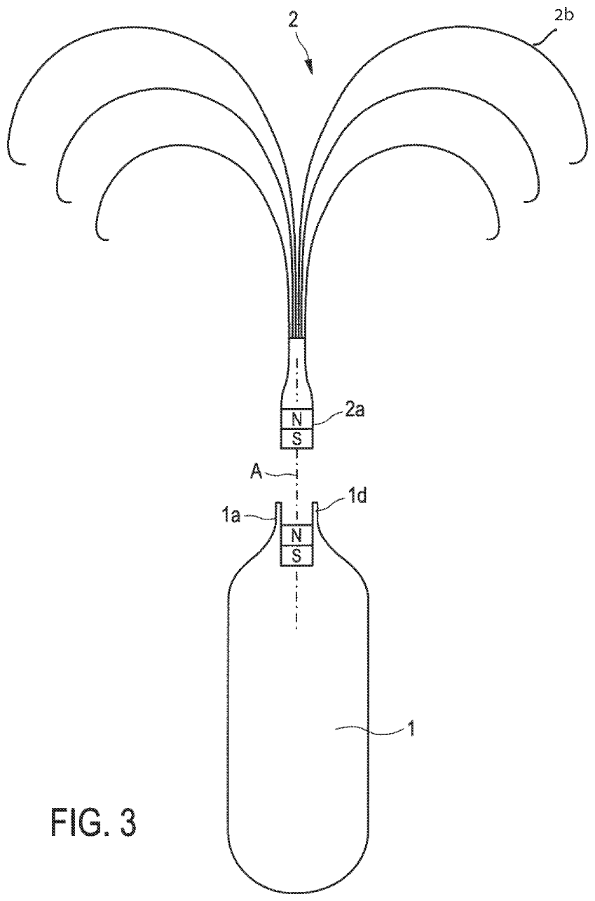

FIG. 3 shows a magnetic link, providing a force link. The connectors 1a and 2a comprise respective permanent magnets, facing each other with opposite polarized surfaces and accordingly the connectors 1a I 2a automatically attract each other. The collar 1d provides an additional form link defining the axis of rotation. The friction between the contacting surfaces prevents unintentional rotation.

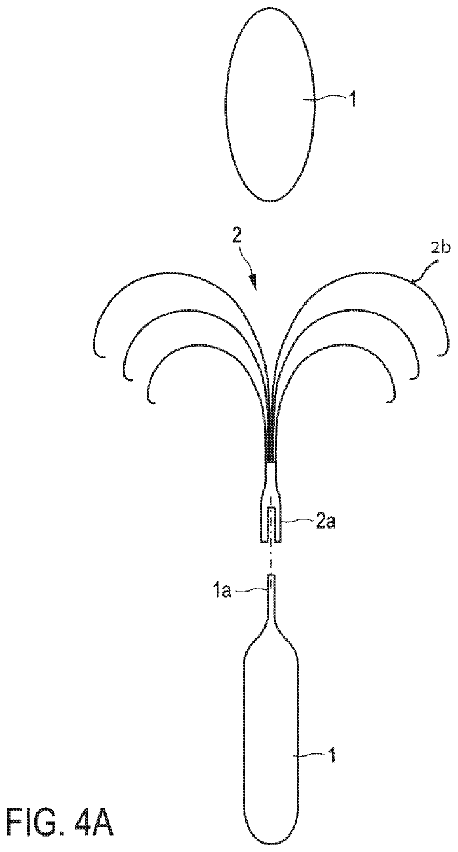

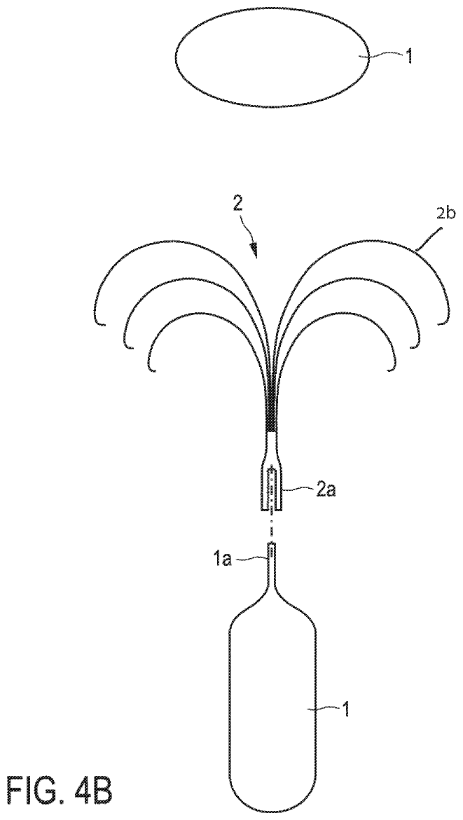

FIGS. 4A-B show a screw-nut arrangement. The closure element 1 comprises a screw as connector 1a and the anchoring element comprises the nut as connector 2a. The connectors are detachable and once they are connected may be adjusted simultaneously in the axial and the rotational positions by turning for example the closure element 1. A friction force in the screw-nut-arrangement may also prevent unintentional movement. It is depicted in FIGS. 4A and 4B that changing the rotational position provides the advantage to allow adjustment of the side of the closure element 1 facing the closing leaflets if the closure element 1 is non-circular in cross section as it is depicted in the upper part of FIGS. 4A and 4B.

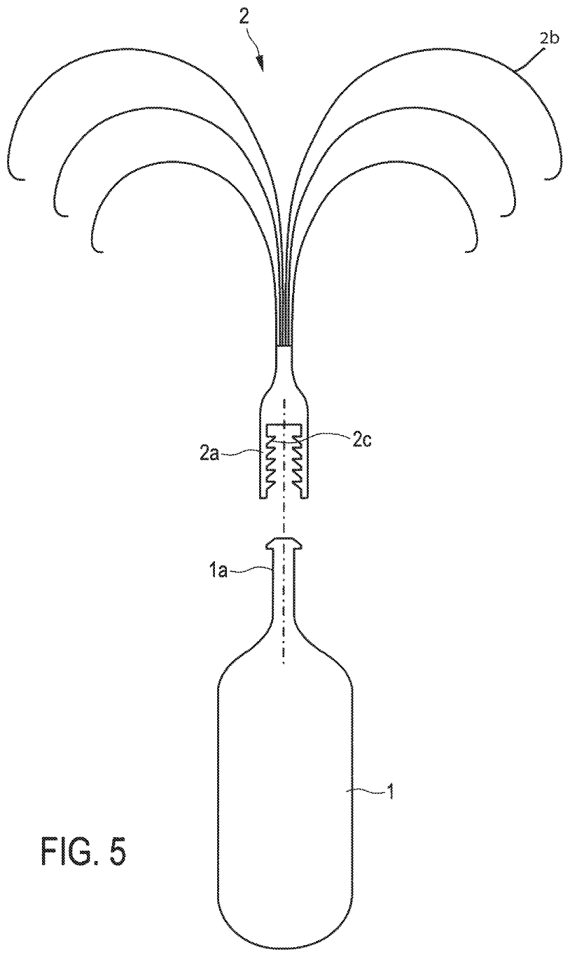

FIG. 5 shows a ratchet arrangement allowing independent adjustment of the axial and the rotational positions. In each stop position defined by the teeth 2c the connector 1a and as such the entire closure element 1 may be rotated without amending the axial position. By selecting a different stop position, the axial position may be changed. Also here a friction lock may be provided to prevent unintentional movement.

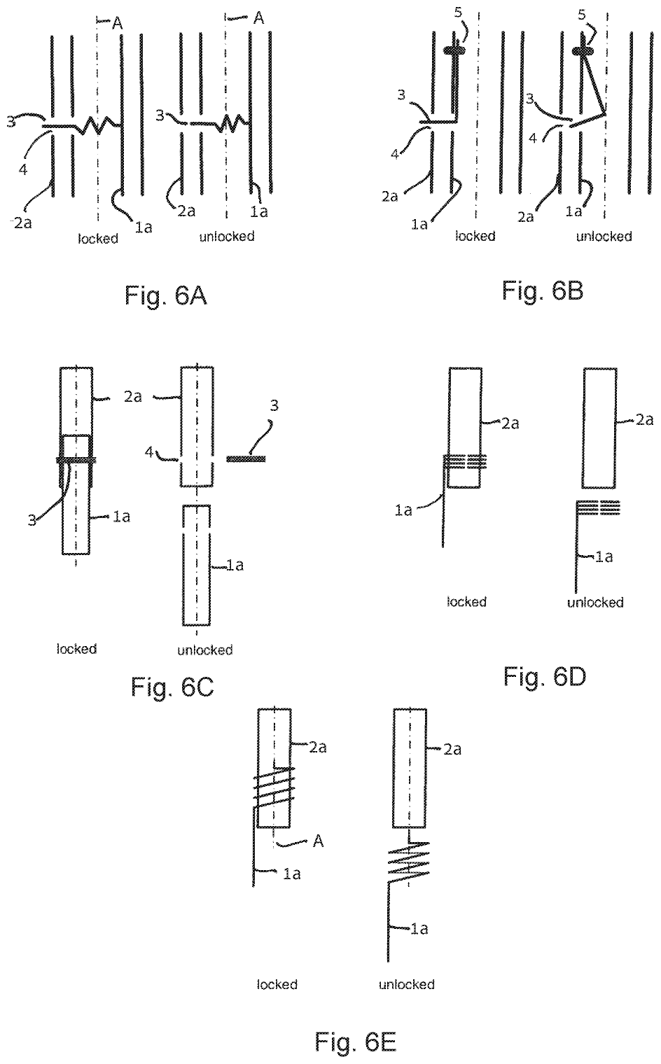

FIGS. 6A-E schematically show other types of connections between the connectors 1a and 2a. The respective closure element and anchoring element are not shown here. It is to be understood that the closure element is attached to the connector 1a and the anchoring element attached to the connector 2a, particularly as shown in the other FIGS. 1 to 5.

The FIGS. 6A-E show the respective locked and unlocked situations of the two connectors 1a and 2a. FIGS. 6A to 6C show embodiments in which the connector 1a is coaxially received in the connector 2a. This may be vice versa as well. At least the receiving connector, here connector 2a is formed as a tube in the receiving section. The received connector, here connector 1a may be a tube but may be solid as well.

In FIG. 6A an embodiment is shown in which connector 1a is coaxially received in connector 2a and secured by a locking mechanism. The locking mechanism is comprising a spring biased locking element 3. The locking mechanism is at least partially situated in the interior of connector 1a and its locking element 3 is protruding in radial direction out of the lateral surface of the connector 1a and through a hole 4 of connector 2a, securing the two connectors against any axial and/or angular movement. The connector 2a may have several holes in different angular and/or axial positions for receiving the locking element 3 in it, thus allowing the adjustment of the two connectors in different angular and/or axial direction, simultaneously locking the chosen position by the engagement of hole 4 and locking element 3. By moving the locking element 3 against its biasing force out of the hole 4 the two connectors 1a and 2a may be detached or another position may be chosen. Such movement may be performed by using a non shown external handle passing through a catheter to the implantation site.

FIG. 6B shows a similar embodiment in which the locking element 3 is also situated at least partially in the connector 1a but secured to it in a hinge connection 5. Also here the locking element 3 may by pushed by a biasing force through the lateral surface of connector 1a and a hole 4 in connector 2a positioned in-line with the locking element 3. Detaching is performed by pushing the locking element 3 back, i.e. out of the hole 4. Also here the connector 2a may have several holes 4 in different angular and/or axial positions for receiving the locking element 3 in it, thus allowing the adjustment of the two connectors in different angular and/or axial direction, simultaneously locking the chosen position by the engagement of hole 4 and locking element 3.

In the embodiments of FIGS. 6A and 6B the locking element is a part being permanently connected to the locking mechanism. In contrast to this FIG. 6C shows an embodiment in which a locking element 3 is a separate part. Also here at least one of the connectors 1a, 2a may have several pairs of opposite lying holes 4. Such pairs may be at different axial and I or angular positions, thus allowing to connect and lock the two connectors in different axial and/or angular positions. The locking element 3 may be a bolt passing through two aligned pairs of holes in the respective connectors.

FIG. 6D shows an embodiment in which the two connectors 1a and 2a are connectable and lockable in different axial I angular positions by means of a friction force link. Connector 2a may by a tube or a solid rod. Connector 1a is formed of one or more rings having a gap in circumferential direction, thus allowing to spread the diameter. The diameter in equilibrium of the ring(s) is chosen so that it is slightly smaller than the outer diameter of connector 2a. Accordingly the diameter needs to be spread for inserting the connector 2a into the ring(s). Such spreading exerts a force to the surface of connector 2a, thus holding the two connectors in place by friction and/or force link. The ring-part of connector 1a may be placed in any desired axial or angular position on the surface or connector 2a.

FIG. 6E shows another embodiment of a force or friction link connection between the two connectors 1a and 2a. In this case connector 2a may be a tube or solid rod. Connector 1a comprises a helically wound spring wire having a diameter in the equilibrium state that is slightly smaller than the outer diameter of connector 2a, as mentioned for the FIG. 6A. Exerting a torque to the spring wire around axis A may enlarge or reduce the diameter, depending upon the direction. If the spring wire is placed on the surface of connector 2a in a desired position the axial or angular movement is inhibited by the exerted force.

In contrast to the FIGS. 6A to 6C the connection between the connectors 1a and 2a in FIGS. 6D and 6E is self-locking by the friction force exerted from connector 1a to the surface of connector 2a. Detaching of the connectors or moving the connectors to a different relative axial and or angular position is performed by overcoming this friction force.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A heart implant for reducing or eliminating a heart valve insufficiency comprising:
a. a closure element being configured to close or reduce a gap between closed valve leaflets when positioned within a valve annulus; and
b. an anchoring element attached to the closure element for fixing the implant in the heart via surface contact between the exterior surface of the anchoring element and an interior surface of a heart lumen;
wherein the closure element and the anchoring element are connected with a mechanism for changing the relative position of the closure element and the anchoring element to each other.

2. The heart implant of claim 1, wherein the mechanism is configured capable of changing the axial position and/or rotational position of the closure element and the anchoring element with respect to each other.

3. The heart implant of claim 1, wherein the mechanism is configured capable of simultaneous changes to axial and rotational positions.

4. The heart implant of claim 1, wherein the mechanism includes an adjustable pair of connectors.

5. The heart implant of claim 4, wherein the pair of connectors are a nut and screw.

6. The heart implant of claim 1, wherein the closure element and the anchoring element are detachable from each other.
